(19) United States
(12) Patent Application Publication
(10) Pub. No.: US 2019/0125174 A1
(43) Pub. Date: May 2, 2019

(54) ELECTRONIC ENDOSCOPE SYSTEM

(71) Applicant: HOYA CORPORATION, Tokyo (JP)

(72) Inventor: Takao MAKINO, Tokyo (JP)

(73) Assignee: HOYA CORPORATION, Tokyo (JP)

(21) Appl. No.: 16/095,645

(22) PCT Filed: Jul. 25, 2017
(86) PCT No.: PCT/IB2017/054488

§ 371 Date: Oct. 22, 2018

(30) Foreign Application Priority Data
Jun. 14, 2016 (JP) 2016-118385

(51) Int. Cl. A61B 1/045; H04N 7/18; G02B 23/24; A61B 1/05

(52) U.S. Cl. CPC A61B 1/045; H04N 7/18; A61B 1/00009; G02B 23/2423; A61B 1/05; G02B 23/2461

(57) ABSTRACT

An electronic endoscope system includes: an imaging element for imaging a subject illuminated alternately with narrow-band light and broadband light; generating, as a first image signal, an image signal of the subject imaged during an illumination period of the narrow-band light; and generating, as a second image signal, an image signal of the subject imaged during an illumination period of the broadband light. The system also includes a signal processing circuit for generating a high-intensity image signal by adding the first and second image signals together; for generating a low-intensity image signal by adding the first and second image signals together after a signal level of the second image signal is reduced and for generating an HDR image signal using the high-intensity and low-intensity image signals.

### TECHNICAL FIELD

The present invention relates to an electronic endoscope system.

### BACKGROUND ART

A known electronic endoscope system generates an HDR (High Dynamic Range) image having an extended dynamic range to clearly display both highlight and shadow areas of a subject. To obtain such an HDR image, a high-intensity image signal obtained by imaging the subject at a high exposure value and a low-intensity image signal obtained by imaging this same subject at a low exposure value need to be combined together. For example, JP 2011-24885A (hereinafter, referred to as “Patent Document 1”) discloses a specific configuration of an electronic endoscope system capable of generating an HDR image.

The electronic endoscope system disclosed in Patent Document 1 includes a light source that alternately changes a light emission time for each field. In a field having a longer light emission time of the light source, the amount of light received by an imaging element is larger. On the other hand, in a field having a shorter light emission time of the light source, the amount of light received by the imaging element is smaller. Thus, a high-intensity image signal is obtained in the former field and a low-intensity image signal is obtained in the latter field. The electronic endoscope system disclosed in Patent Document 1 generates the HDR image using these image signals.

### SUMMARY OF INVENTION

Recent years have seen electronic endoscope systems that generate an image by narrow-band imaging by which a specific living organism is enhanced using narrow-band light having high absorption characteristics with respect to the specific living organism. Typically, the narrow-band light is obtained by filtering white light emitted from a white light source into narrow half-width light using an optical filter. On this account, the narrow-band light has an extremely smaller amount of light than the white light. Thus, clear imaging of the subject with the narrow-band light is difficult, and it is also difficult to obtain the high-intensity image signal necessary to generate the HDR image.

The present invention is conceived in view of the circumstances described above and has an object to provide an electronic endoscope system suitable for generating an HDR image including an enhanced image of a specific living organism of a subject illuminated with narrow-band light.

An electronic endoscope system according to an aspect of the present invention includes; a light source unit which alternately emits narrow-band light and broadband light; a generation means for imaging a subject illuminated alternately with the narrow-band light and the broadband light, generates, as a first image signal, an image signal of the subject imaged during an illumination period of the narrow-band light, and generates, as a second image signal, an image signal of the subject imaged during an illumination period of the broadband light; a high-intensity image signal generation means for generating a high-intensity image signal by adding the first image signal to the second image signal; a low-intensity image signal generation means for generating a low-intensity image signal by adding the first image signal to the second image signal after a signal level of the second image signal is reduced by multiplying the second image signal by a predetermined coefficient; and an HDR (High Dynamic Range) image signal generation means for generating an HDR image signal using the high-intensity image signal and the low-intensity image signal.

Moreover, according to an aspect of the present invention, the high-intensity image signal generation means and the low-intensity image signal generation means may generate the high-intensity image signal and the low intensity image signal, respectively using the first image signal and the second image signal of the subject imaged during illumination periods temporally adjacent to each other.

Furthermore, according to an aspect of the present invention, the predetermined coefficient may be one of a constant and a value that is set based on a signal level ratio between the first image signal and the second image signal.

An aspect of the present invention provides an electronic endoscope system suitable for generating an HDR image including an enhanced image of a specific living organism of a subject illuminated with narrow-band light.

### BRIEF DESCRIPTION OF DRAWINGS

FIG. 1 is a block diagram showing a configuration of an electronic endoscope system according to an embodiment of the present invention.

FIG. 2 is a front view of a rotary filter unit provided for a processor and viewed from a condenser lens, according to an embodiment of the present invention.

FIG. 3 is a flowchart of a signal processing operation performed in an HDR mode by a signal processing circuit provided for the processor according to an embodiment of the present invention.

FIG. 4 is a diagram conceptually illustrating processing of generating a high-intensity image signal according to an embodiment of the present invention.

FIG. 5 is a diagram conceptually illustrating processing of generating a low-intensity image signal according to an embodiment of the present invention

### DESCRIPTION OF EMBODIMENTS

Hereinafter, an embodiment according to the present invention is described, with reference to the drawings. In the following, an electronic endoscope system is described as an example according to the embodiment of the present invention.

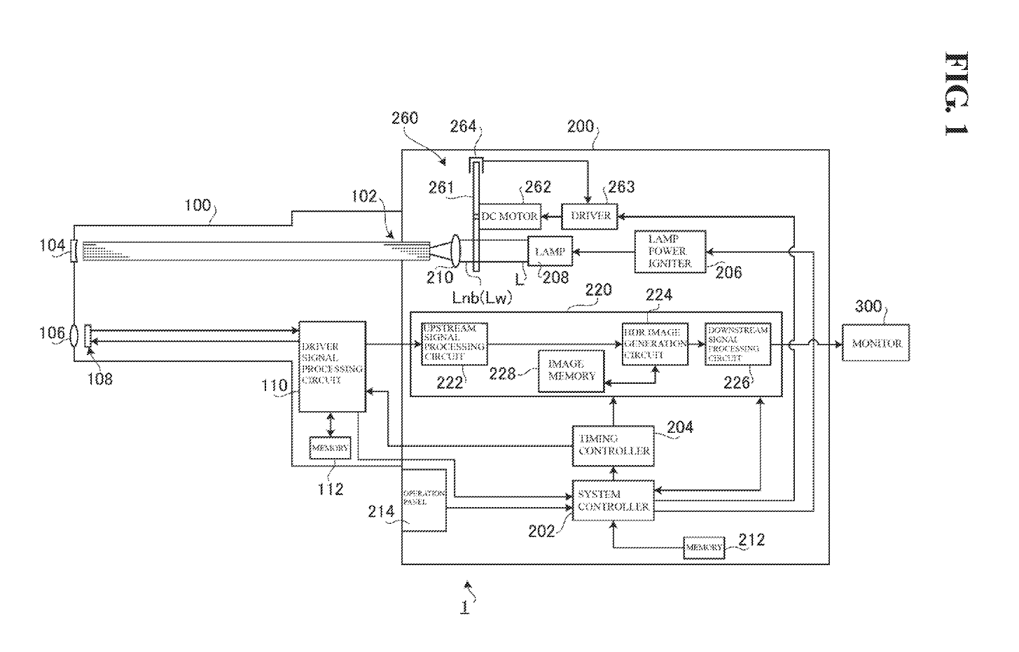

FIG. 1 is a block diagram showing a configuration of an electronic endoscope system 1 according to the embodiment of the present invention. As shown in FIG. 1, the electronic endoscope system 1 is a system specialized for medical use and includes an electronic scope 100, a processor 200, and a monitor 300.

The processor 200 includes a system controller 202 and a timing controller 204. The system controller 202 executes various programs stored in a memory 212 and comprehensively control the whole of the electronic endoscope system 1.

Moreover, the system controller 202 is connected to an operation panel 214. In response to an instruction inputted by an operator through the operation panel 214, the system controller 202 executes operations of the electronic endoscope system 1 and changes parameters for the operations. Examples of the instruction inputted by the operator include an instruction to switch an operation mode of the electronic endoscope system 1. The operation mode includes a normal mode and an HDR mode, for example. The timing controller 204 outputs, to each circuit of the electronic endoscope system 1, a clock pulse to adjust the timing of operation performed by the corresponding unit.

Upon being actuated by a lamp power igniter 206, a lamp 208 emits illumination light L. The lamp 208 is a high-intensity lamp, such as a xenon lamp, a halogen lamp, a mercury lamp, or a metal halide lamp. The lamp 208 may be a semiconductor light-emitting element, such as an LD (Laser Diode) or an LED (Light Emitting Diode). The illumination light L is light (white light) including at least a visible light region.

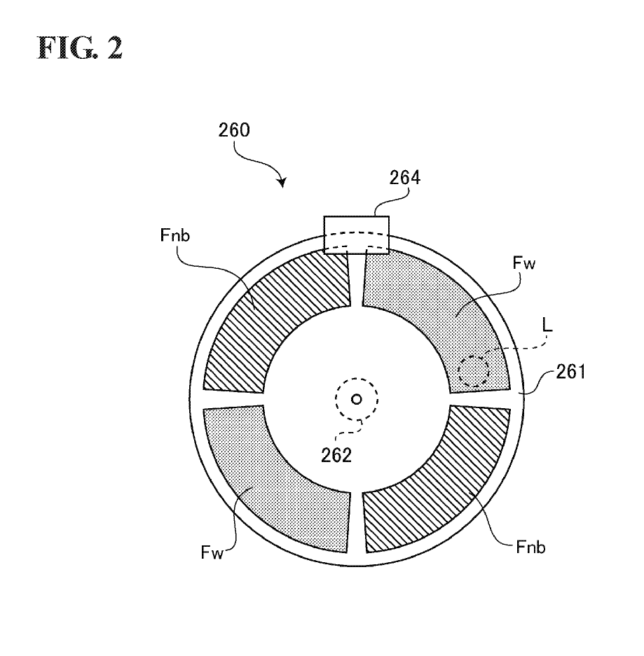

The illumination light L emitted from the lamp 208 enters a rotary filter unit 260. FIG. 2 is a front view of the rotary filter unit 260 seen from a condenser lens 210. The rotary filter unit 260 includes a rotating turret 261, a DC motor 262, a driver 263, an a photo-interrupter 264.

As shown in FIG. 2, a narrow-band light filter Fnb and a white light filter Fw are alternately arranged in the rotating turret 261 in a circumferential direction. Each of these optical filters has the shape of a sector. The optical filters are arranged with an angular pitch corresponding to a frame period (about 90° of angular pitch in this example). In the following description, the “frame” may also be referred to as the “field”.

The driver 263 drives the DC motor 262 under the control of the system controller 202. The DC motor 262 causes the rotating turret 261 to make a rotary motion, and this allows the rotary filter unit 260 to extract, from the illumination light L incident from the lamp 208, one of two types of illumination light (narrow-band light Lnb and white light Lw) different in the spectrum, at a timing synchronized with imaging.

To be more specific, during the rotary motion, the rotating turret 261 alternately extracts the narrow-band light Lnb from the narrow-band light filter Fnb and broadband light (i.e., the white light Lw) having a broader band than the narrowband light Lnb from the white light filter Fw. A rotary position and a rotary phase of the rotating turret 261 are controlled through the photo-interrupter 264 detecting an aperture (not shown) formed near an outer region of the rotating turret 261.

The narrow-band filter Fnb has spectral characteristics suitable for taking an image by narrow-band imaging by which an image of a specific living organism (such as a vascular structure in a superficial or deeper layer or a specific lesion area) is enhanced. After passing through the narrow-band light filter Fnb, the illumination light L becomes narrow half-width light, that is, the narrow-band light Lnb, having high absorption characteristics with respect to the specific living organism.

The white light filter Fw is a neutral-density filter that reduces the amount of light of the illumination light L to an appropriate amount. It should be noted that the white light filter Fw may be replaced with a simple aperture (having no optical filter) or a slit (having no optical filter) combined with a diaphragm function.

The illumination light extracted by the rotary filter unit 260 (that is, the narrow-band light Lnb or the white light Lw) is condensed by the condenser lens 210 on an incident end face of an LCB (Light Carrying Bundle) 102 of the electronic scope 100 and then enters the LCB 102.

The illumination light (the narrow-band light Lnb or the white light Lw) entering the LCB 102 passes through the LCB 102 and is emitted from an emission end face of the LCB 102 positioned at a tip of the electronic scope 100. Then, the emitted illumination light illuminates a living tissue, which is the subject, inside the body cavity via a light distributing lens 104. As a result, the living tissue is illuminated alternately with the narrow-band light Lnb and the white light Lw. Here, return light from the living tissue illuminated with the illumination light forms an optical image on a light receiving surface of a solid-state imaging element 108 via an objective lens 106.

The solid-state imaging element 108 is a single-plate color CCD (Charge Coupled Device) image sensor having a Bayer pixel arrangement. The solid-state imaging element 108 accumulates the optical image formed by pixels on the light receiving surface, as electric charge corresponding to the amount of light. Then, the solid-state imaging element 108 generates and outputs an image signal for each of R (Red), G (Green), and B (Blue). Note that the solid-state imaging element 108 is not limited to the CCD image sensor, and may be replaced with a CMOS (Complementary Metal Oxide Semiconductor) image sensor or an imaging device of a different type. Moreover, the solid-state imaging element 108 may include a complementary color filter.

The timing for the rotary filter unit 260 to switch between the narrow-band light Lnb and the white light Lw is synchronized with the timing for the solid-state imaging element 108 to switch an imaging period (a frame period). Thus, in one frame period, the solid-state imaging element 108 receives the return light from the living tissue illuminated with the narrow-band light Lnb to generate and output the image signal of the image by narrow-band imaging. Then, in a subsequent frame period, the solid-state imaging element 108 receives the return light from the living tissue illuminated with the white light Lw to generate and output the image signal of the image by white-light imaging. By repetitions of the above process, the solid-state imaging element 108 alternately outputs the image signal obtained by narrow-band imaging and the image signal obtained by white-light imaging.

A driver signal processing circuit 110 is provided in a connection unit of the electronic scope 100. Each of the image signals obtained by narrow-band imaging and white-light imaging is inputted into the driver signal processing circuit 110 from the solid-state imaging element 108 in a frame period. The driver signal processing circuit 110 performs predetermined processing on the image signal inputted from the solid-state imaging element 108 and outputs the resulting signal to a signal processing circuit 220 of the processor 200.

Furthermore, the driver signal processing circuit 110 accesses a memory 112 to read out specific information on the electronic scope 100. The specific information on the electronic scope 100 that is recorded in the memory 112 includes, for example, the number of pixels, sensitivity, achievable frame rate, and model number of the solid-state imaging element 108. The driver signal processing circuit 110 outputs the specific information read from the memory 112 to the system controller 202.

On the basis of the specific information on the electronic scope 100, the system controller 202 performs various operations to generate a control signal. Using the generated control signal, the system controller 202 controls operations and timings of the circuits in the processor 200 so that processing appropriate to the electronic scope connected to the processor 200 is performed.

The timing controller 204 supplies a clock pulse to the driver signal processing circuit 110 according to the timing control performed by the system controller 202. In response to the clock pulse supplied from the timing controller 204, the driver signal processing circuit 110 performs drive control on the solid-state imaging element 108 in synchronization with the frame rate of video processed in the processor 200.

The signal processing circuit 220 includes an upstream signal processing circuit 222, an HDR image generation circuit 224, a downstream signal processing circuit 226, and an image memory 228. A signal processing operation performed by the signal processing circuit 220 is described for each of the operation modes of the electronic endoscope system 1, that is, the normal mode and the HDR mode.

Case where Operation Mode is Set to Normal Mode

The upstream signal processing circuit 222 performs demosaic processing, a matrix operation, and predetermined signal processing, such as Y/C separation, on each of the image signals obtained by narrow-band imaging and white-light imaging and alternately inputted from the driver signal processing circuit 110 in a frame period. Then, the upstream signal processing circuit 222 outputs the resulting signal to the HDR image generation circuit 224.

The HDR image generation circuit 224 outputs, without performing processing, each of the image signals obtained by narrow-band imaging and white-light imaging and alternately inputted from the upstream signal processing circuit 222 in a frame period, to the downstream signal processing circuit 226.

The downstream signal processing circuit 226 performs processing on each of the image signals obtained by narrow-band imaging and white-light imaging and alternately inputted from the HDR image generation circuit 224 in a frame period, to generate screen data for monitor display. Then, the downstream signal processing circuit 226 converts the generated screen data for monitor display into a predetermined video format signal. The video format signal obtained by conversion is outputted to the monitor 300. As a result, the image of the living tissue obtained by narrow-band imaging and white-light imaging is displayed on a display screen of the monitor 300.

Case where Operation Mode is Set to HDR Mode

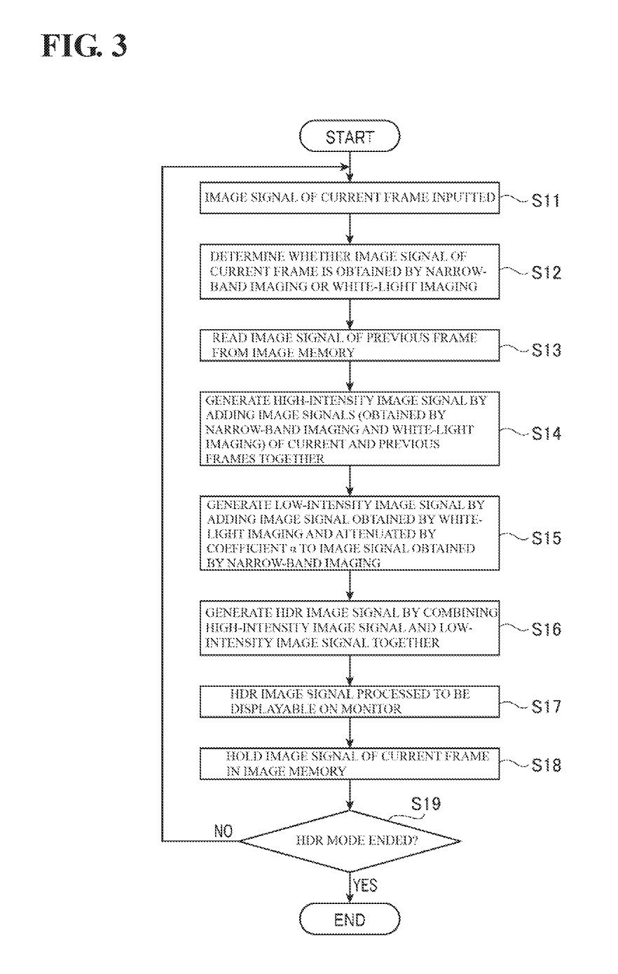

FIG. 3 is a flowchart of a signal processing operation performed by the signal processing circuit 220 in the HDR mode. The flowchart shown in FIG. 3 is started when, for example, the operation mode of the electronic endoscope system 1 is switched to the HDR mode.

S11 in FIG. 3 (Input of Image Signal of Current Frame)

In this processing step S11, the image signal of the current frame (that is, the image signal obtained by narrow-band imaging or by white-light imaging) is inputted to the upstream signal processing circuit 222.

S12 in FIG. 3 (Determination of image Signal)

In this processing step S12, the HDR image generation circuit 224 determines whether the image signal of the current frame that is inputted from the upstream signal processing circuit 222 in the processing step S11 (Input of Image Signal of Current Frame) is the image signal obtained by narrow-band imaging or by white-light imaging. For example, the HDR image generation circuit 224 determines whether the image signal of the current frame is obtained by narrow-band imaging or by white-light imaging, on the basis of: the information on the control performed by the system controller 202 on the rotary filter unit 260 and so forth; and a value of the image signal, such as an average luminance value.

S13 in FIG. 3 (Reading of Image Signal of Previous Frame)

The image memory 228 (volatile memory) holds the image signal of the previous frame (the frame one frame before the current frame) by execution of a processing step S18 (Holding of Image Signal of Current Frame) described later. In this processing step S13, the HDR image generation circuit 224 reads the image signal of the previous frame from the image memory 228. Here, when the image signal of the current frame is the signal obtained by narrow-band imaging, the image signal obtained by white-light imaging is read out. When the image signal of the current frame is the signal obtained by white-light imaging, the image signal obtained by narrow-band imaging is read out.

Here, suppose that the operation mode is set to the HDR mode at the startup of the electronic endoscope system 1. In this case, the image signal of the previous frame is not held in the image memory 228 because the processing shown in this flowchart is executed for the first time. Thus, in this case, the processing of this flowchart proceeds to the processing step S18 (Holding of image Signal of Current Frame) described later.

S14 in FIG. 3 (Generation of High-Intensity Image Signal)

In this processing step S14, the HDR image generation circuit 224 generates a high-intensity image signal by adding the image signal of the current frame to the image signal of the previous frame read out in the processing step S13 (Reading of Image Signal of Previous Frame).

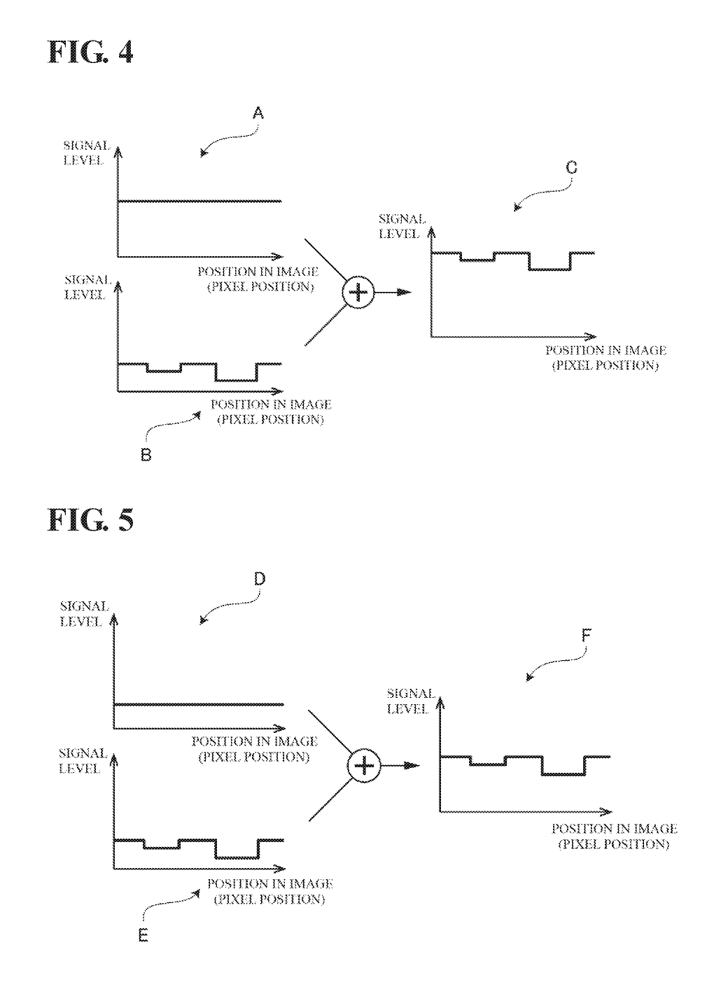

FIG. 4 is a diagram conceptually illustrating processing of generating a high-intensity image signal. A graph A in FIG. 4 conceptually shows signal levels (luminance values) of pixels forming the image signal obtained by white-light imaging. For example, the graph A in FIG. 4 shows the signal levels of the pixels representing a surface part, such as mucosa. A graph B in FIG. 4 conceptually shows signal levels of pixels forming the image signal obtained by narrow-band imaging. For example, the graph B in FIG. 4 shows the signal levels of the pixels representing a specific living organism in addition to the surface part, such as mucosa. In the graph B, two recessed parts correspond to the pixels representing the specific living organism and parts other than these recessed parts correspond to the pixels representing, for example, the mucosa. Thus, the graph B includes information on the specific living organism.

In the example shown in FIG. 4, the image signal obtained by white-light imaging (see the graph A in FIG. 4) is added to the image signal obtained by narrow-band imaging (see the graph B in FIG. 4). As shown in a graph C in FIG. 4, this addition increases the signal level of the image signal obtained by narrow-band imaging by the added level (by the signal level of the image signal obtained by white-light imaging) while maintaining the information on the specific living organism. In this way, an image signal with high intensity, that is, a high-intensity image signal is obtained.

S15 in FIG. 3 (Generation of Low-Intensity Image Signal)

In this processing step S15, when the image signal of the current frame is determined in the processing step S12 (Determination of Image Signal) as being the image signal obtained by white-light imaging, the HDR image generation circuit 224 multiplies the image signal of the current frame by a coefficient α. On the other hand, when the image signal of the current frame is determined in the aforementioned step as being the image signal obtained by narrow-band imaging, the HDR image generation circuit 224 multiplies the image signal of the previous frame (i.e., the image signal obtained by white-light imaging) by the coefficient α.

The coefficient α is a value smaller than 1. For this reason, the multiplication by the coefficient α reduces (attenuates) the signal level of the image signal obtained by white-light imaging. In this processing step S15, by the addition of the image signal obtained by white-light imaging that is multiplied by the coefficient α to the image signal obtained by narrow-band imaging, a low-intensity image signal is generated.

FIG. 5 is a diagram conceptually illustrating processing of generating a low-intensity image signal. A graph D in FIG. 5 conceptually shows signal levels of pixels forming the image signal obtained by white-light imaging. The signal levels in the graph D in FIG. 5 are obtained by multiplying the signal levels of the pixels shown in the graph A in FIG. 4 by the coefficient α. As can be seen from the graph D in FIG. 5, the multiplication of the image signal obtained by white-light imaging by the coefficient α reduces the signal level and causes the image signal to have low intensity. Here, the graph D in FIG. 5 is the same as the graph B in FIG. 4.

In the example shown in FIG. 5, the image signal obtained by white-light imaging (see the graph D in FIG. 5) is added to the image signal obtained by narrow-band imaging (see a graph E in FIG. 5). As shown in a graph F in FIG. 5, this addition increases the signal level of the image signal obtained by narrow-band imaging by the slightly added level (by the signal level of the image signal obtained by white-light imaging that is multiplied by the coefficient α) while maintaining the information on the specific living organism. In this way, an image signal with low intensity, that is, a low-intensity image signal is obtained.

The coefficient α is a constant or a variable. In the latter case, the coefficient α is a learning value, for example, and is periodically updated based on a signal level ratio (such as an average value ratio) between the image signals of two previous consecutive frames (the image signal by narrow-band imaging and the image signal by white-light imaging). The smaller the signal level ratio is (the smaller the difference between the image signal by narrow-band imaging and the image signal by white-light imaging is), the smaller the value of the coefficient α is set to ensure the signal level difference between the high-intensity image signal and the low-intensity image signal.

S16 in FIG. 3 (Generation of HDR Image Signal)

The high-intensity image signal generated in the processing step S14 (Generation of High-Intensity Image Signal) is suitable for reproducing the information on the living tissue that is too dark with blocked-up shadows. Moreover, the low-intensity image signal generated in the processing step S15 (Generation of Low-Intensity Image Signal) is suitable for reproducing the information on the living tissue that is too bright with blown-out highlights. In the processing step S16, the HDR image generation circuit 224 combines the high-intensity image signal and the low-intensity image signal having the above-described characteristics, to generate an HDR image signal having an extended dynamic range. It should be noted that the technology of combining the high-intensity image signal and the low-intensity image signal to generate the HDR image signal is well known, and that the detailed description on this technology is thus omitted here.

S17 in FIG. 3 (Display Processing on HDR Image)

In this processing step S17, the HDR image signal generated in the processing step S16 (Generation of HDR Image Signal) is inputted into the downstream signal processing circuit 226. The downstream signal processing circuit 226 converts the received HDR image signal into the predetermined video format signal and then outputs this video format signal to the monitor 300. As a result, the image of the living tissue by narrow-band imaging is displayed with a wide dynamic range on the display screen of the monitor 300.

Here, the image signals of the two frames are used to generate the HDR image signal, and this combination (i.e., the combination of the high-intensity image signal and the low-intensity image signal) is updated for each frame. On this account, the HDR image is displayed on the display screen of the monitor 300, with no change in the frame rate.

S18 in FIG. 3 (Holding of Image Signal of Current Frame)

In this processing step S18, the HDR image generation circuit 224 holds, in the image memory 228, the image signal of the current frame that is inputted from the upstream signal processing circuit 222 in the processing step S11 (Input of Image Signal of Current Frame).

S19 in FIG. 3 (Determination on End of HDR Mode)

In this processing step 519, whether imaging of the living tissue in the HDR mode is ended is determined by determining, for example, whether the operation mode is switched to a different mode. When it is determined that imaging of the living tissue in the HDR mode is not ended (S19: NO), the processing proceeds to the processing step S11 (Input of Image Signal of Current Frame) in this flowchart. When it is determined that imaging of the living tissue in the HDR mode is ended (S19: YES), the processing of this flowchart is terminated.

According to the present embodiment, the image obtained by narrow-band imaging is increased in intensity using the image signal obtained by white-light imaging, so that the high-intensity image signal is generated. With this, the HDR image including the information on the specific living organism can be generated, which has conventionally been difficult.

The exemplary embodiment according to the present invention has been described thus far. Embodiments according to the present invention are not limited to the embodiment described above. Various modifications can be made within the technical idea according to the present invention. For example, an exemplary embodiment according to the present invention can be implemented by appropriately combining exemplary embodiments clearly described in the specification or obvious embodiments.

In the embodiment described above, the high-intensity image signal and the low-intensity image signal are generated using the image signals obtained in the illumination periods temporally adjacent to each other (that is, the current frame and the immediately previous frame). In another embodiment, the high-intensity image signal and the low-intensity image signal may be generated using the image signals obtained in the illumination periods temporally separated from each other (for example, the current frame and the frame before the immediately previous two frames).

1-3. (canceled)

4. An electronic endoscope system, comprising:
a light source unit configured to alternately emit narrow-band light and broadband light;
an imaging element configured to image a subject illuminated alternately with the narrow-band light and the broadband light; to generate, as a first image signal, an image signal of the subject imaged during an illumination period of the narrow-band light; and to generate, as a second image signal, an image signal of the subject imaged during an illumination period of the broadband light;
a signal processing circuit configured to generate a high-intensity image signal by adding the first image signal to the second image signal; to generate a low-intensity image signal by adding the first image signal to the second image signal after a signal level of the second image signal is reduced by multiplying the second image signal by a predetermined coefficient; and to generate a high dynamic range (HDR) image signal using the high-intensity image signal and the low-intensity image signal.

5. The electronic endoscope system according to claim 4, wherein the light source unit comprises a lamp and a plurality of filters.

6. The electronic endoscope system according to claim 4, wherein the imaging element comprises an image sensor.

7. The electronic endoscope system according to claim 4,
wherein the signal processing circuit is configured to generate the high-intensity image signal and the low-intensity image signal, respectively, using the first image signal and the second image signal of the subject imaged during illumination periods temporally adjacent to each other.

8. The electronic endoscope system according to claim 7,
wherein the predetermined coefficient is one of:
a constant; and
a value set based on a signal level ratio between the first image signal and the second image signal.

9. The electronic endoscope system according to claim 4,
wherein the predetermined coefficient is one of:
a constant; and
a value set based on a signal level ratio between the first image signal and the second image signal.

10. A method of generating an image of a specific living organism, the method comprising:
alternately emitting narrow-band light and broadband light;
imaging a subject illuminated alternately with the narrow-band light and the broadband light;
generating, as a first image signal, an image signal of the subject imaged during an illumination period of the narrow-band light;
generating, as a second image signal, an image signal of the subject imaged during an illumination period of the broadband light;
generating a high-intensity image signal by adding the first image signal to the second image signal;
generating a low-intensity image signal by adding the first image signal to the second image signal after a signal level of the second image signal is reduced by multiplying the second image signal by a predetermined coefficient; and
generating an HDR image signal using the high-intensity image signal and the low-intensity image signal.

11. The method according to claim 10, wherein the alternately emitting narrow-band light and broadband light comprises alternately arranging a plurality of filters with respect to illumination light incident from a lamp.

12. The method according to claim 10, wherein the subject is a living tissue inside a body cavity.

13. The method according to claim 10,
wherein the generating the high-intensity image signal and the low-intensity image signal, respectively, comprise using the first image signal and the second image signal of the subject imaged during illumination periods temporally adjacent to each other.

14. The method according to claim 13,
wherein the predetermined coefficient is one of:
a constant; and
a value set based on a signal level ratio between the first image signal and the second image signal.

15. The method according to claim 10,
wherein the predetermined coefficient is one of:
a constant; and
a value set based on a signal level ratio between the first image signal and the second image signal.
